(19) United States
(12) Patent Application Publication
(10) Pub. No.: US 2016/0271411 A1
(43) Pub. Date: Sep. 22, 2016

(54) COLD PLASMA PRESSURE TREATMENT SYSTEM

(71) Applicant: Plasmology4, Inc., Scottsdale, AZ (US)

(72) Inventors: Robert M. Hummel, Cave Creek, AZ (US); David J. Jacofsky, Peoria, AZ (US); Marc C. Jacofsky, Phoenix, AZ (US); Jeffrey I. Meyers, Phoenix, AZ (US); Steven A. Myers, Scottsdale, AZ (US)

(21) Appl. No.: 15/072,263

(22) Filed: Mar. 16, 2016

Related U.S. Application Data
(60) Provisional application No. 62/134,388, filed on Mar. 17, 2015.

(51) Int. Cl. A61N 1/44; A61H 9/00

(52) U.S. Cl. CPC A61N 1/44; A61H 9/0057; A61H 2201/10; A61H 2201/5007; A61H 2201/165

(57) ABSTRACT

A system including a cold plasma pressure treatment system including a pump configured to produce negative pressure at a first cold plasma treatment region, and a controller configured to control negative pressure at the first cold plasma treatment region with the pump and an electrical signal that forms cold plasma.

### CROSS-REFERENCE TO RELATED APPLICATIONS

This application claims priority to and benefit of U.S. Provisional Application No. 62/134,388 entitled “Cold Plasma Pressure Treatment System,” filed on Mar. 17, 2015, which is hereby incorporated by reference in its entirety.

### BACKGROUND

This section is intended to introduce the reader to various aspects of art that may be related to various aspects of the present invention, which are described and/or claimed below. This discussion is believed to be helpful in providing the reader with background information to facilitate a better understanding of the various aspects of the present invention. Accordingly, it should be understood that these statements are to be read in this light, and not as admissions of prior art.

Modern medicine enables physicians to treat a wide variety of wounds and infections. For example, physicians may treat these wounds and infections using topical medication (e.g., creams, foams, gels, ointments, bandages, etc.) and/or internal medication (e.g., medicine administered orally, intravenously). Unfortunately, existing treatments may be costly, ineffective, and/or slow to treat certain wounds and infections.

### BRIEF DESCRIPTION OF THE DRAWINGS

Various features, aspects, and advantages of the present invention will become better understood when the following detailed description is read with reference to the accompanying figures in which like characters represent like parts throughout the figures, wherein:

FIG. 1 is a front view of an embodiment of a cold plasma pressure treatment system coupled to a patient;

FIG. 2 is a schematic of an embodiment of a cold plasma pressure treatment system with a wearable cold plasma applicator;

FIG. 3 is a schematic of an embodiment of a cold plasma pressure treatment system with a wearable cold plasma applicator;

FIG. 4 is a cutaway perspective view of an embodiment of a conduit coupled to a wearable cold plasma applicator;

FIG. 5 is a cross-sectional view of an embodiment of a conduit coupled to a wearable cold plasma applicator along line 5-5 of FIG. 4;

FIG. 6 is a cross-sectional view of an embodiment of electrodes in a conduit taken within line 6-6 of FIG. 4;

FIG. 7 is a cross-sectional view of an embodiment of electrodes in a conduit taken within line 6-6 of FIG. 4;

FIG. 8 is a cutaway perspective view of an embodiment of a conduit coupled to a wearable cold plasma applicator;

FIG. 9 is a cutaway perspective view of an embodiment of a conduit coupled to a wearable cold plasma applicator;

FIG. 10 is a cross-sectional side view of a wearable cold plasma applicator using a fluid-based dielectric barrier discharge (DBD) coupled to a conduit; and

FIG. 11 is a cross-sectional side view of an embodiment of a wearable cold plasma applicator using a cascade DBD coupled to a conduit.

### DETAILED DESCRIPTION OF SPECIFIC EMBODIMENTS

One or more specific embodiments of the present invention will be described below. These described embodiments are only exemplary of the present invention. For example, some or all of the drawings may include exaggerated features, sizes, distances, etc. to facilitate comprehension of the present invention. Additionally, in an effort to provide a concise description of these exemplary embodiments, all features of an actual implementation may not be described in the specification. It should be appreciated that in the development of any such actual implementation, as in any engineering or design project, numerous implementation-specific decisions must be made to achieve the developers' specific goals, such as compliance with system-related and business-related constraints, which may vary from one implementation to another. Moreover, it should be appreciated that such a development effort might be complex and time consuming, but would nevertheless be a routine undertaking of design, fabrication, and manufacture for those of ordinary skill having the benefit of this disclosure.

The disclosed embodiments include a cold plasma pressure treatment system that includes a wearable cold plasma applicator. In operation, the cold plasma pressure treatment system facilitates healing of open wounds with negative pressure wound therapy, while simultaneously providing a cold plasma treatment. It should be understood that the cold plasma pressure treatment system is capable of generating cold plasma in variety of pressure conditions including atmospheric pressure and sub-atmospheric pressure. In some embodiments, the cold plasma pressure treatment system uses negative pressure wound therapy (e.g., sub-atmospheric pressure) to remove excess fluids, cellular waste, infectious material, etc. from the wound bed, while the cold plasma accelerates healing by killing bacteria, reducing inflammation, increasing blood coagulation, etc. As will be explained below, the cold plasma pressure treatment system couples to a patient with the wearable cold plasma applicator to focus the treatment on areas of interest (e.g., a treatment site, such as an open wound). In some embodiments, the wearable cold plasma applicator may conform to the shape of the wound/infection site (e.g., arm, leg, chest, hand, neck, etc.) while providing one or more apertures that couple to a conduit to enable a pump to provide negative pressure (e.g., a sub-atmospheric vacuum) at the wound/infection site. For example, the wearable cold plasma applicator may be formed into a sleeve, patch, bandage, glove, shoe, garment, sheet, etc. enabling targeted and uniform treatment of an entire wound or infection site.

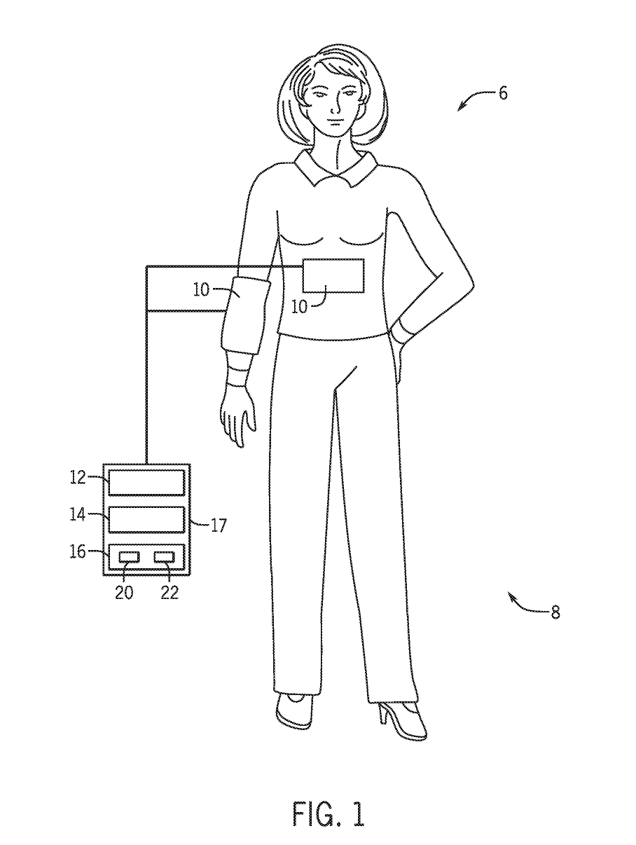

FIG. 1 is a front view of an embodiment of a patient 6 with a cold plasma pressure treatment system 8 (e.g., medical treatment system). The cold plasma pressure treatment system 8 may include a wearable cold plasma applicator 10, a pump 12 (e.g., a vacuum pump), a gas source 14, and a controller 16. As illustrated, the cold plasma pressure treatment system 8 may combine the pump 12, gas source 14, and controller 16 into an integrated module or housing 17 (e.g., portable and/or wearable unit) that can be carried by a patient 6 during activities or for use in an environment away from a medical facility. In portable embodiments, the housing 17 may include a power source (e.g., battery, photovoltaic cells, crank powered generator, power outlet, etc.) that enables the cold plasma pressure treatment system 8 to operated in a wide variety of locations. In other embodiments the pump 12, gas source 14, and controller 16 may not be integrated while still providing the same treatment. In operation, the pump 12 may remove fluid (e.g., gas or liquid) from or around a wound site on the patient 6 by creating negative pressure on or around the wound bed. In other words, the pump 12 acts as a suction device pulling excess fluids, cellular waste, infectious material, etc. from the wound bed to facilitate/accelerate healing. In addition to removing materials from the wound bed, the cold plasma pressure treatment system 8 may form cold plasma around the wound site to stimulate healing by killing bacteria, accelerating blood coagulation, stimulating the release of growth factors, etc. The cold plasma may be formed using atmospheric air next to the wound bed or using a specialized working gas from the gas source 14. In some embodiments, the specialized gas plasma may form specific ions that focus on killing bacteria or that promote faster healing (e.g., combinations of helium, oxygen, OH ions). The gas may be a specific gas or mixture of gases (e.g., helium, neon, argon, krypton, xenon, radon, oxygen, nitrogen, or any combination thereof) that form cold plasmas with different properties ideally suited for specific treatments (e.g., a gas that promotes faster wound healing, blood coagulation, infection treatment, etc.).

In operation, the cold plasma pressure treatment system 8 uses the controller 16 to produce an electrical signal that ionizes the gas (e.g., gas from gas source 14, atmospheric gases) converting it into a plasma. For example, the controller 16 may send the electrical signal to the wearable cold plasma applicator 10 enabling the wearable cold plasma applicator 10 to produce cold plasma from atmospheric gases next to the patient's skin, or with gas from the gas source 14. In some embodiments, the cold plasma pressure treatment system 8 may form cold plasma in one or more conduits or lumens coupled to the wearable cold plasma applicator 10. In still other embodiments, the cold plasma pressure treatment system 8 may form the cold plasma within the housing 17 (e.g., form cold plasma within the pump 12, form cold plasma within a cold plasma generating chamber).

As illustrated, the controller 16 includes one or more processors 20 and one or more memories 22. In operation, the controller 16 uses the processor 20 to execute instructions stored in the memory 22 to produce and control the cold plasma generating electrical signal (e.g., change power, amplitude, frequency/frequencies, pulse timing, etc.). In some embodiments, the electrical signal may be a multi-frequency, harmonic-rich signal (e.g., a timed pulse electrical signal that is pulsed between 100-1000 Hz with an output voltage between 1-100 KV peak-peak having multiple A/C waves at multiple frequencies that overlap to produce 2-2,000,000 or more harmonic components between DC and 500 MHz). As the multi-frequency, harmonic-rich electrical signal passes through the gas (e.g., gas from the gas source 14 or atmospheric gases); the gas molecules/atoms lose and gain electrons to produce cold plasma with positive ions, negative ions, and electrons. It is believed that the multi-frequency, harmonic-rich electrical signal facilitates removal of electrons from molecules/atoms with less energy than typical plasma formation. Accordingly, the plasma is a low temperature plasma or cold plasma (e.g., a cold plasma with a temperature between approximately 60-120, 60-80, 70-90, 80-100, 90-110, 100-120 degrees Fahrenheit), enabling exposure to a temperature sensitive target substrate (e.g., biological tissue). Furthermore, and as will be explained in more detail below, the controller 16 may simultaneously control the pump 12 to create negative pressure at the wound site as well as the release of gas from the gas source 14, by executing instructions stored in the memory 22 with the processor 20.

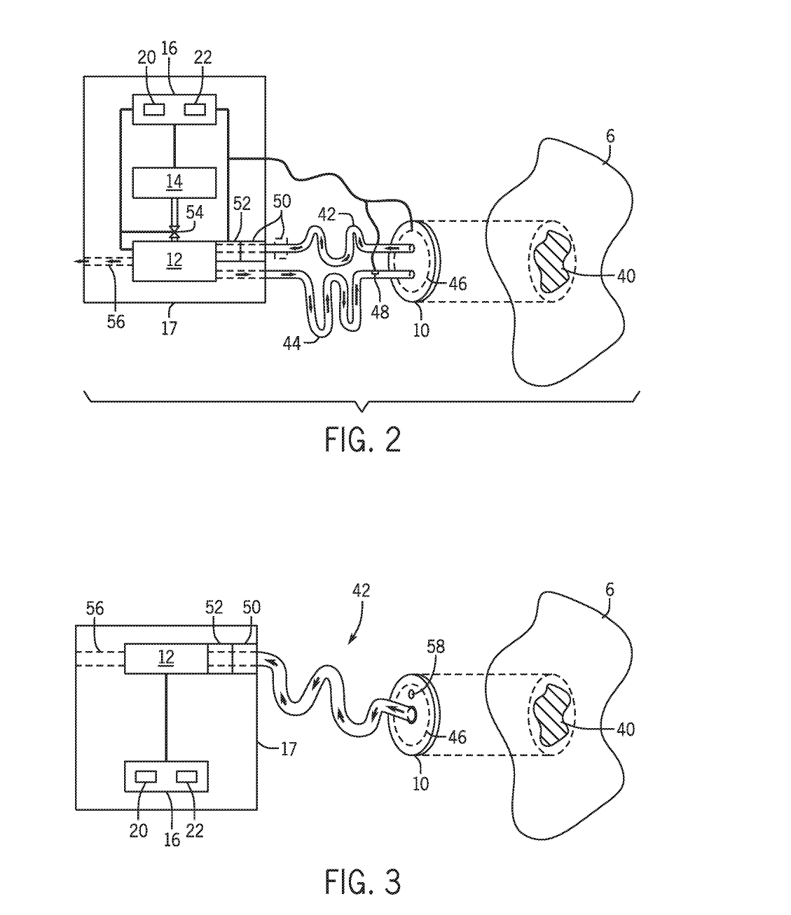

FIG. 2 is a schematic of an embodiment of a cold plasma pressure treatment system 8 capable of treating a wound site 40 (e.g., open wound, wound bed). In operation, the wearable cold plasma applicator 10 couples to the patient 6 around the wound site 40. The wearable cold plasma applicator 10 may be made out of a flexible dielectric material that enables the wearable cold plasma applicator 10 to couple to the patient 6 forming a seal around the wound site 40. For example, the wearable cold plasma applicator may be made out of silicone, latex, open cell foam, gauze, hydrogels, polyoxymethylene, polyamide, polytetrafluoroethylene (PTFE), acetal homopolymer, polyethylene (PE), polypropylene (PP), poly vinyl chloride (PVC), ethylene vinyl acetate (EVA), propylene, copolyester ether, and polyolefin film, among others.

Coupled to the wearable cold plasma applicator 10 are one or more return conduits 42 (e.g., 1, 2, 3, 4, 5, or more) that enable the pump 12 to remove (e.g., draw, vacuum, or suction) material (e.g., fluids, cellular waste, infectious material, etc.) from the wound site 40 to facilitate/accelerate healing. As the pump 12 removes material from the wound site 40, the pump 12 may draw gas from the gas source 14 through one or more gas supply conduits 44 (e.g., 1, 2, 3, 4, 5, or more) to the wearable cold plasma applicator 10. In some embodiments, the return and supply conduits 42, 44 may be integral with the wearable cold plasma applicator 10 or couple to the wearable cold plasma applicator 10 (e.g., snap fit, compression fit, threaded connection, etc.). The gas flowing through the supply conduit 44 then passes through the wearable cold plasma applicator 10 where the gas may be converted into a cold plasma before returning to the pump 12 through the return conduit 42. For example, the wearable cold plasma applicator 10 may include an electrode 46 that receives the electrical signal from the controller 16. As the electrode 46 receives the electrical signal, the electrode 46 generates cold plasma with the gas in and around the wound site 40. In some embodiments, the supply conduit 44 may include one or more electrodes 48 that enable cold plasma generation in the gas supply conduit 44, which is then drawn by the pump 12 over the wound site 40. In other embodiments, the cold plasma pressure treatment system 8 may generate and combine cold plasma formed within the supply conduit 44 with the electrodes 48, cold plasma generated by the electrode 46, and/or with generating cold plasma within the housing 17.

The cold plasma pressure treatment system 8 may have several control modes executable by the controller 16. For example, the control modes may include preset parameters for controlling the pump 12 (e.g., to provide continuous, pulsing, gradually increasing, gradually decreasing, and/or patterns of negative pressure treatment) and/or the generation of cold plasma (e.g., starting, stopping, and/or continuous application of cold plasma). These modes may be preprogrammed for certain types of wounds (e.g., burn, infection, cut, etc.) and their severity. Other control modes may include recirculating gas through the wearable cold plasma applicator 10, venting the gas after use around the wound site 40, or a combination of venting and recirculating the gas. For example, in the recirculation mode, the pump 12 draws material and gas through the return conduit 42 to the pump 12. The pump 12 then channels the gas back into the supply conduit 44 where the gas is drawn back to the wearable cold plasma applicator 10 by negative pressure. In embodiments that recirculate gas, the cold plasma pressure treatment system 8 may include a filter and/or collection container 50 that collects liquids, infectious materials, cell waste, etc. while still enabling the gas to continue through to the pump 12. The filter and/or collection container 50 (e.g., bag or canister) may be located within the housing 17 or placed inline with the conduit 42. Some embodiments may include more than one filter and/or collection container 50 (e.g., 1, 2, 3, 4, 5, or more). For example, some filters and/or collection containers 50 may be located within and/or external to the housing 17. The cold plasma pressure treatment system 8 may also include one or more plasma chambers 52. In operation, the cold plasma chamber 52 disinfects any liquids that may have passed through the filter(s) and/or collection container 50 by reconverting the gas into a cold plasma with an electrical signal from the controller 16. The pump 12 then recirculates the gas back into the supply conduit 44. However, even in the recirculation mode, the cold plasma pressure treatment system 8 may lose gas (e.g., through conduit connections, through the wearable cold plasma applicator 10, etc.). Accordingly, the controller 16 may measure the amount of gas passing through the pump 12, or otherwise determine the need for more gas, and periodically open a valve 54 to enable additional gas from the gas source 14 to enter the supply conduit 44. As will be appreciated, operating in the recirculation mode may conserve gas and reduce power consumption by the controller 16. For example, in some embodiments, once the gas is first ionized, subsequent ionizations may demand less power input from the controller 16.

In a vent mode, the cold plasma pressure treatment system 8 may continuously draw gas from the gas source 14 as the pump 12 pulls gas and materials away from the wound site 40. For example, the housing 17 may include a vent port 56 that enables the pump 12 to push previously used gas out of the cold plasma pressure treatment system 8. However, this gas may still be filtered with the filter and/or container 50 and disinfected in the cold plasma chamber 52. Finally, the cold plasma pressure treatment system 8 may operate in a hybrid mode that combines recirculation and venting of the gas. For example, the controller 16 may periodically vent gas out of the cold plasma pressure treatment system 8 based on a time of usage, number of ionizations, or other relevant guidelines. In other embodiments, the controller 16 may continuously vent a percentage of the previously used gas (e.g., 5%, 10%, 15%, 20%, 25% or more) while replacing the vented percentage with an equivalent percentage of fresh gas from the gas source 14. Regardless of the operating mode, the controller 16 monitors the negative pressure created by the pump 12 to ensure that the negative pressure remains within a threshold range (e.g., 400 mmHG-750 mmHG, 450 mmHg-500 mmHg, 500 mmHg-550 mmHg, 550 mmHg-600 mmHg, 600 mmHg-650 mmHg, 650 mmHg-700 mmHg, 700 mmHg-750 mmHg), this ensures that the wound site 40 is properly drained while protecting the wound site 40 from further injury. In some embodiments, the controller 16 may control the pump 12 to periodically increase and decrease the negative pressure to increase circulation to the wound site 40.

FIG. 3 is a schematic of an embodiment of a cold plasma pressure treatment system 8 capable of treating a wound site 40 (e.g., open wound, wound bed). In operation, the wearable cold plasma applicator 10 couples to the patient 6 around the wound site 40. The wearable cold plasma applicator 10 may be made out of a flexible dielectric material that enables the wearable cold plasma applicator 10 to couple to the patient 6 forming a seal around the wound site 40. In contrast, to the cold plasma pressure treatment system 8 in FIG. 2, the cold plasma pressure treatment system 8 in FIG. 3 may not include a gas source within the housing 17. Instead, the cold plasma pressure treatment system 8 may use atmospheric air surrounding the wound site 40 to generate cold plasma for treatment (e.g., killing bacteria, improving blood coagulation). For example, the cold plasma applicator 10 may include one or more openings or valves 58 (e.g., one-way valves) that enable atmospheric air to pass through the cold plasma applicator 10 to the wound site for cold plasma generation. In some embodiments, the valve or orifice 58 may reduce the flow rate of atmospheric gases through the cold plasma applicator 10 to a flow rate less than the removal flow rate of the pump 12, thus enabling the pump 12 to maintain negative pressure on the wound site 40.

For example, the wearable cold plasma applicator 10 may include an electrode 46 that receives the cold plasma generating electrical signal from the controller 16. As the electrode 46 receives the electrical signal, the electrode 46 generates cold plasma with the atmospheric gases surrounding the wound site 40. While the electrode 46 generates cold plasma around the wound site 40, the pump 12 draws material and gas away from the wound site 40 through a return conduit 42. The conduit(s) 42 may be integral with the wearable cold plasma applicator 10 or couple to the wearable cold plasma applicator 10 (e.g., snap fit, compression fit, threaded connection, etc.). As illustrated, the cold plasma pressure treatment system 8 may include a single return conduit 42. However, in some embodiments there may be more than one return conduit 42 (e.g., 1, 2, 3, 4, 5, or more) that enable the pump 12 to remove (e.g., draw, vacuum, or suction) material (e.g., fluids, cellular waste, infectious material, etc.) from the wound site 40 to facilitate/accelerate healing. The conduit 42 conducts the material and gas to the housing 17 and through a filter and/or container 50. The filter and/or container 50 collects liquids, infectious materials, cellular waste, etc. before venting the gas out of the pump 12 through the vent port 56. Some embodiments may include more than one filter and/or container 50 (e.g., 1, 2, 3, 4, 5, or more) with filters and/or containers 50 located within and external to the housing 17 as well as a cold plasma chamber 52. As the pump 12 draws material and gas away from the wound site 40, fresh atmospheric gas may pass through the wearable cold plasma applicator 10 or through gaps between the wearable cold plasma applicator 10 and the patient 6.

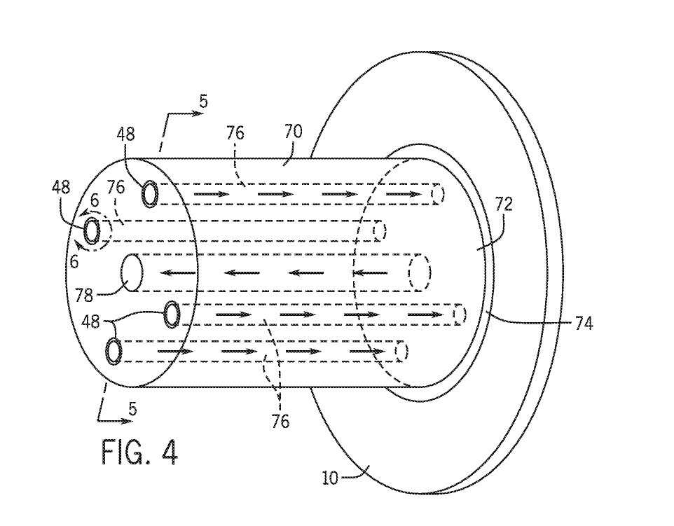

FIG. 4 is a cutaway perspective view of an embodiment of a conduit 70 coupled to a wearable cold plasma applicator 10. In some embodiments, the cold plasma pressure treatment system 8 may include a single conduit 70 for the supply and return of material and gas, instead of return conduit 42 and supply conduit 44. As illustrated, the conduit 70 couples to an aperture 72 in the wearable cold plasma applicator 10 at a connection 74. The connection 74 may be a snap fit, compression fit, threaded connection, etc. Once connected, the conduit 70 enables fluid to flow to and from the wearable cold plasma applicator 10. For example, the conduit 70 may include one or more supply lumens 76 (e.g., 1, 2, 3, 4, 5, or more) and one or more return lumens 78 (e.g., 1, 2, 3, 4, 5, or more). In operation, the supply lumens 76 channel gas from the gas source 14 through the wearable cold plasma applicator 10 to the wound site 40. Along the way, the gas may be converted into cold plasma for treatment. For example, the supply lumens 76 may include electrodes 48 that enable cold plasma formation within the supply lumens 76. In some embodiments, the electrodes 48 may act as a false ground to maintain previously generated cold plasma in an ionized state as the cold plasma flows to the wound site 40. In still other embodiments, the electrodes 48 may be a combination of electrodes that form cold plasma and false grounds that maintain the cold plasma after formation. As cold plasma flows to the wound site 40, the return lumen 78 enables the pump 12 to maintain a negative pressure at the wound site, which draws/removes material and gas away from the wound site 40 to facilitate healing.

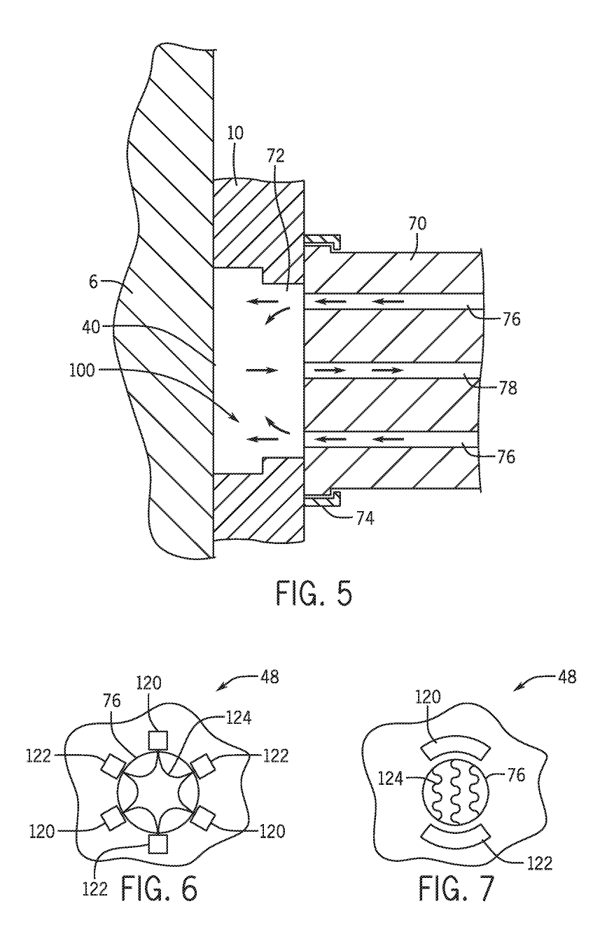

FIG. 5 is a cross-sectional view of an embodiment of a conduit 70 coupled to a wearable cold plasma applicator 10, taken along line 5-5 of FIG. 4. As illustrated, the wearable cold plasma applicator 10 couples to the gas source 14 with conduit 70. The conduit 70 in turn couples to the wearable cold plasma applicator 10 with the connection 74 (e.g., friction fit, snap fit, thread coupling, quick-release coupling, etc.). After passing through the supply lumens 76, the cold plasma enters a plasma treatment region 100 (e.g., medical treatment region). As the cold plasma passes through the plasma treatment region 100 toward ground (e.g., patient 6), the cold plasma contacts the wound site 40 killing bacteria and stimulating the healing process (e.g., blood coagulation). While the supply lumen 76 supplies the gas used to form the cold plasma, the return lumen 78 simultaneously draws the gas and other materials away from the wound site 40 to facilitate healing.

FIGS. 6 and 7 are cross-sectional views of a supply lumen 76 through the conduit 70 within line 6-6 of FIG. 4. As illustrated, the conduit 70 may include multiple electrodes 48 that surround the supply lumen 76. The electrodes 48 may be powered electrodes 120 and/or grounded electrodes 122. In operation, the powered electrodes 120 receive an electrical signal from the controller 16. Once a sufficient amount of charge builds on the powered electrode 120, the electrical signal (e.g., multi-frequency harmonic-rich electrical signal) crosses through the gas in the lumen 76 to the grounded electrode 122, forming cold plasma 124. As illustrated in FIG. 6, there may be multiple electrodes 120, 122 (e.g., 2, 3, 4, 5, 6, 7, 8, 9, 10 or more) or only the two seen in FIG. 7. Moreover, in some embodiments with multiple electrodes 120 and 122, the electrodes 120 and 122 may be placed circumferentially about each supply lumen 76 along the length or partial length of the conduit 70. Furthermore, instead of including powered electrodes 120 and grounded electrodes 122, some embodiments may include only grounded electrodes 122 or only powered electrodes 120. For example, grounded electrodes 122 may attract previously formed cold plasma by providing a ground image for the plasma, reducing cold plasma dissipation as cold plasma travels through the supply lumen 76.

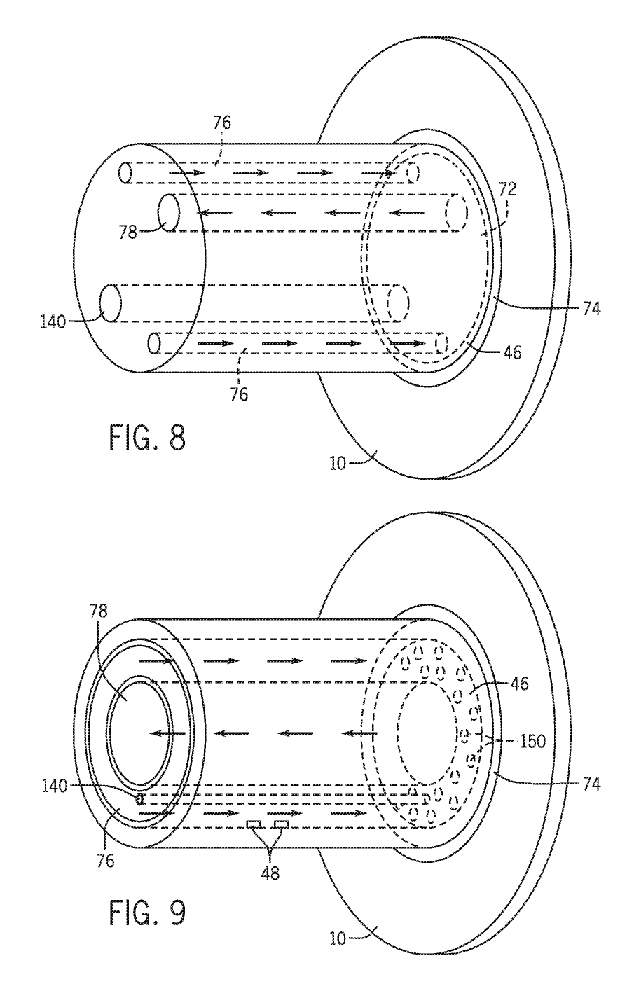

FIG. 8 is a cutaway perspective view of an embodiment of a conduit 70 coupled to a wearable cold plasma applicator 10. As illustrated, the cold plasma pressure treatment system 8 includes the conduit 70 for the supply and return of material and gas (e.g., instead of the return conduit 42 and a supply conduit 44 seen in FIG. 2). As illustrated, the conduit 70 couples to an aperture 72 in the wearable cold plasma applicator 10 at a connection 74. The connection 74 may be a snap fit, compression fit, threaded connection, etc. Once connected, the conduit 70 enables fluid flow to and from the wearable cold plasma applicator 10. For example, the conduit 70 may include one or more supply lumens 76 (e.g., 1, 2, 3, 4, 5, or more) and one or more return lumens 78 (e.g., 1, 2, 3, 4, 5, or more). In operation, the supply lumens 76 channel gas from the gas source 14 through the wearable cold plasma applicator 10 to the wound site 40. After passing through the supply lumens 76, the gas enters the cold plasma treatment region 100 where the electrode 46 ionizes the gas with the electrical signal (e.g., multi-frequency, harmonic-rich electrical signal) passing through a high-voltage cable 140. As explained above, while the supply lumen 76 supplies the gas used to form the cold plasma, the return lumen 78 simultaneously draws the gas and other materials away from the wound site 40 to facilitate healing.

FIG. 9 is a cutaway perspective view of an embodiment of a conduit 70 coupled to a wearable cold plasma applicator 10. As illustrated, the cold plasma pressure treatment system 8 includes the conduit 70 coupled to the wearable cold plasma applicator 10. As illustrated, the conduit 70 couples to an aperture 72 in the wearable cold plasma applicator 10 at a connection 74. The connection 74 may be a snap fit, compression fit, threaded connection, etc. Once connected, the conduit 70 enables fluid flow to and from the wearable cold plasma applicator 10. For example, the conduit 70 may include one or more supply lumens 76 (e.g., 1, 2, 3, 4, 5, or more) and one or more return lumens 78 (e.g., 1, 2, 3, 4, 5, or more) in a coaxial arrangement within a respective supply lumen 76 (e.g., concentric). In operation, the supply lumens 76 channel gas from the gas source 14 to the wearable cold plasma applicator 10 and through apertures 150 in the electrode 46 to the wound site 40. After passing through the electrode 46, the gas enters the cold plasma treatment region 100 where the electrode 46 ionizes the gas converting it into cold plasma. In some embodiments, the multi-frequency harmonic-rich electrical signal may pass through the high-voltage cable 140 within the conduit 70 to the electrode 46. In still other embodiments, the conduit 70 may include electrodes 48 that enable plasma generation within the supply lumen 76. As explained above, while the supply lumen 76 supplies the gas used to form the cold plasma, the return lumen 78 simultaneously draws the gas and other materials away from the wound site 40 to facilitate healing.

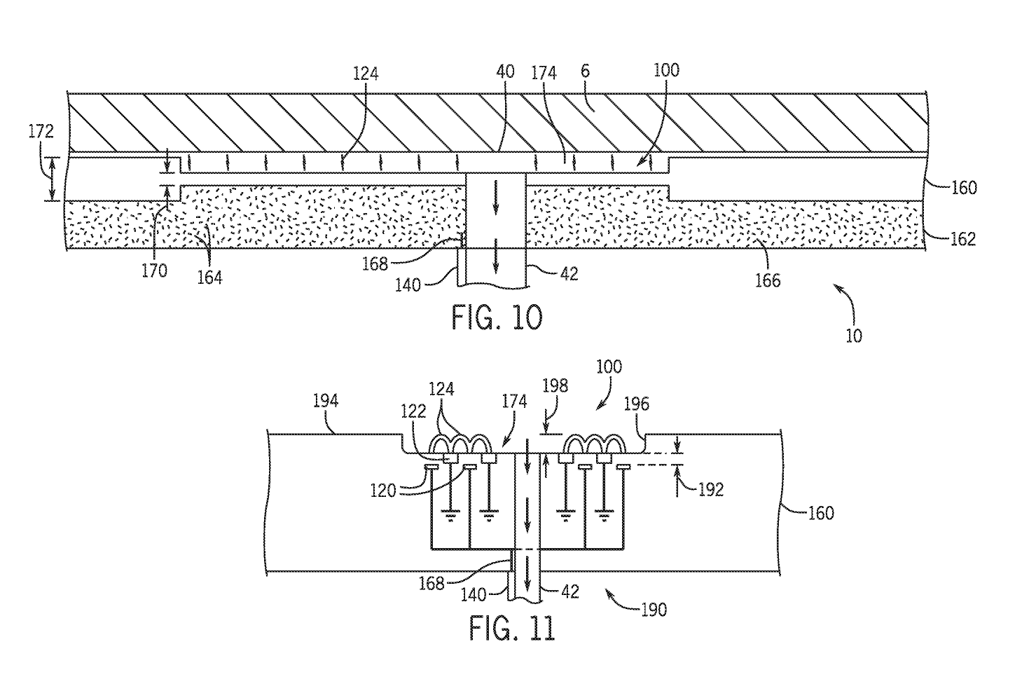

FIG. 10 is a cross-sectional side view of a wearable cold plasma applicator 10 using a fluid-based dielectric barrier discharge (DBD) coupled to a return conduit 42. As explained above, the wearable cold plasma applicator 10 may include a flexible dielectric barrier layer 160 (e.g., silicone, latex, open cell foam, gauze, hydrogels, polyoxymethylene, polyamide, polytetrafluoroethylene (PTFE), acetal homopolymer, polyethylene (PE), polypropylene (PP), poly vinyl chloride (PVC), ethylene vinyl acetate (EVA), propylene, copolyester ether, and polyolefin film) coupled to flexible fluid filled layer 162. In some embodiments, the fluid filled layer 162 may be a multi-phase fluid that includes conductive material 164 in a fluid 166 (e.g., gas and/or liquid). The flexibility of the dielectric barrier layer 160 and the fluid filled layer 162 enables the wearable cold plasma applicator 10 to conform to the shape of a patient's body 6 for more effective treatment on a variety of patients and anatomical sites.

In operation, the electrical signal from the controller 16 passes through the cable 140 (e.g., HV/RF feed cables) to the conductive wire electrode 168 (e.g., tungsten) in the flexible fluid filled layer 162. As the electrical signal enters the flexible fluid filled layer 162, the fluid 166 conducts the electrical signal through the flexible fluid filled layer 162 to the cold plasma generation region 100. As illustrated, the flexible dielectric barrier layer 160 has a thickness 170 next to the cold plasma generation region 100, while the rest of the dielectric barrier layer 160 has a thickness 172 that is greater than the thickness 170. It is in this cold plasma generation region 100, where the dielectric barrier layer 160 has the thickness 170, that charge is able to build before crossing the air gap 174. In other words, the dielectric barrier layer 160 has a thickness of 172 to reduce or block charge movement and direct it primarily through the cold plasma generation region 100. Once a sufficient amount of charge builds on the dielectric barrier layer 160, the multi-frequency, harmonic-rich electrical signal crosses the air gap 174 to the wound site 40 (e.g., ground), forming the cold plasma 124. As cold plasma 124 treats the wound site 40, the pump 12 draws fluids and materials from the wound site 40 through the conduit 42. In some embodiments, the cold plasma applicator 10 may include a porous foam (e.g., open cell foam) in the air gap 174. In operation, the foam may collect fluids from the wound site 40 as well as maintain an appropriate distance between the cold plasma applicator 10 and the wound site 40 for cold plasma generation. In some embodiment, the foam may contain substances that are activated and/or carried by the cold plasma to the wound site 40 to facilitate healing.

FIG. 11 is a cross-sectional side view of an embodiment of a wearable cold plasma applicator 10 using a cascade DBD coupled to the conduit 42. The cold plasma applicator 10 may also use a flexible dielectric barrier layer 160 (e.g., silicone, latex, open cell foam, gauze, hydrogels, polyoxymethylene, polyamide, polytetrafluoroethylene (PTFE), acetal homopolymer, polyethylene (PE), polypropylene (PP), poly vinyl chloride (PVC), ethylene vinyl acetate (EVA), propylene, copolyester ether, and polyolefin film). The flexibility of the dielectric barrier layer 160 enables the wearable cold plasma applicator 10 to conform to a patient 6 for more effective treatment on a variety of patients and anatomical sites. However, instead of a flexible fluid filled layer 162 that conducts the electrical signal, the wearable cold plasma applicator 10 of FIG. 11 includes a cascade dielectric barrier discharge (DBD) system 190 embedded in the dielectric barrier layer 160. The cascade DBD system 190 includes a wire electrode 168 (e.g., tungsten, copper) within the cable 140 that provides power to the powered electrodes 120, which enables the wearable cold plasma applicator 10 to form cold plasma in the air gap 174.

In operation, the controller 16 produces the cold plasma generating electrical signal that travels through the cable 140 (e.g., HV/RF feed cables) and the wire electrode 168 (e.g., tungsten) to the powered electrodes 120. As illustrated, the powered electrodes 120 are a distance 192 away from the top surface 194 of the dielectric barrier layer 160, enabling charge to build on the electrode 120. In some embodiments, the powered electrodes 120 may not be covered with dielectric; but instead, may be exposed to the air gap 174. Once a sufficient amount of charge builds on the electrode 120, the electrical signal (e.g., multi-frequency, harmonic-rich electrical signal) crosses through the air gap 174 to the grounded electrodes 122, forming cold plasma 124. To ensure a sufficient air gap 174 between the patient 6 and the wearable cold plasma applicator 10, the dielectric barrier layer 160 may have a recess 196 in the top surface 194. The depth 198 of the recess 196 provides a sufficient amount of atmospheric air for cold plasma formation, while simultaneously enabling the cold plasma 124 to contact the wound site 40 for treatment. As explained above, as the cold plasma is generated during the treatment, the pump 12 draws fluids and materials from the wound site 40 through the conduit 42. In some embodiments, the cold plasma applicator 10 may include a porous foam (e.g., open cell foam) in the air gap 174. In operation, the foam may collect fluids from the wound site 40 as well as maintain an appropriate distance between the cold plasma applicator 10 and the wound site 40 for cold plasma generation. In some embodiment, the foam may contain substances that are activated and/or carried by the cold plasma to the wound site 40 to facilitate healing.

While the invention may be susceptible to various modifications and alternative forms, specific embodiments have been shown by way of example in the drawings and have been described in detail herein. However, it should be understood that the invention is not intended to be limited to the particular forms disclosed. Rather, the invention is to cover all modifications, equivalents, and alternatives falling within the spirit and scope of the invention as defined by the following appended claims.

1. A system comprising:
a cold plasma pressure treatment system, comprising:
a pump configured to produce negative pressure at a first cold plasma treatment region; and
a controller configured to control negative pressure at the first cold plasma treatment region with the pump and an electrical signal that forms cold plasma.

2. The system of claim 1, comprising a gas source coupled to the pump, and wherein the pump is configured to deliver gas from the gas source to the first plasma treatment region.

3. The system of claim 2, comprising a first lumen configured to carry the gas from the pump to the first plasma treatment region.

4. The system of claim 3, comprising a second lumen configured to carry the gas from the first plasma treatment region to the pump.

5. The system of claim 2, wherein the pump is configured to recirculate the gas through the first plasma treatment region.

6. The system of claim 2, wherein the pump discharges the gas after the gas passes through the first plasma treatment region.

7. The system of claim 1, comprising a second plasma treatment region configured to treat the gas after the gas passes through the first plasma treatment region.

8. The system of claim 4, comprising a wearable cold plasma applicator configured to couple to and deliver the cold plasma to a user mountable side.

9. The system of claim 8, wherein the wearable cold plasma applicator comprises open cell foam.

10. A system comprising:
a wearable cold plasma applicator configured to couple to and deliver a cold plasma to a first cold plasma treatment region; and
a cold plasma pressure treatment system coupled to the wearable cold plasma applicator, comprising:
a pump configured to produce a negative pressure at the first cold plasma treatment region; and
a controller configured to control the negative pressure at the first cold plasma treatment region with the pump and an electrical signal that forms cold plasma.

11. The system of claim 10, wherein the cold plasma pressure treatment system comprises at least one conduit that couples to an aperture in the wearable cold plasma applicator.

12. The system of claim 11, wherein the at least one conduit comprises a first lumen that enables the pump to pump fluid away from the first cold plasma treatment region.

13. The system of claim 12, wherein the at least one conduit comprises a second lumen configured to deliver a gas to the first cold plasma treatment region.

14. The system of claim 13, wherein the at least one conduit includes electrodes around the second lumen, and the electrodes are configured to generate the cold plasma as the gas flows through the second lumen.

15. The system of claim 11, wherein the wearable cold plasma applicator comprises electrodes in a flexible dielectric barrier material that generates cold plasma.

16. The system of claim 15, wherein the at least one conduit comprises a cable that delivers the electrical signal to the electrodes.

17. The system of claim 12, wherein cold plasma pressure treatment system comprises a filter configured to filter the fluid passing through the first lumen.

18. A method comprising:
pumping fluid away from a first plasma treatment region;
producing an electrical signal with a controller; and
generating a cold plasma using the electrical signal.

19. The system of claim 18, comprising filtering fluid from the first plasma treatment region.

20. The system of claim 18, comprising generating cold plasma at a second plasma treatment region to treat fluid from the first plasma treatment region.
